SEQUENCE LISTING (2 sequences)

SEQ ID NO: 1 - DNA, 6208 bp, synthetic construct
  misc_feature 1..6208: note = Description of sequence: AAV vector
  source 1..6208: mol_type = other DNA
cgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctcgatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcaacgcgttggatccaccggtgaggagatctgccgccgcgatcgccatgctctcggccaccaggagggccctccagctctttcacagcctcttccccatcccgagaatgggtgactccgccgccaagatcgtcagcccccaggaagccttgccgggccggaaggagcccctcgtcgtagcggccaaacatcatgtcaatggcaacagaacagttgaacctttcccagagggaacacagatggctgtatttggaatgggctgtttctggggagctgaaaggaaattctggaccctgaaaggtgtatattcaactcaagttggttttgcaggaggctatactcccaatcctacttataaagaagtctgctcaggaaaaactggccacgcagaagtagtccgggtggtgttccagccagaacacatcagctttgaggaactgctcaaggtcttctgggagaatcatgacccgacccaaggcatgcgccaaggcaatgaccacggctctcagtaccgctcagccatctacccgacctctgccgagcacgtgggggcagccctgaagtccaaggaggactaccagaaggttctgtcggagcatggtttcggcctgatcaccacggacatccgggagggacaaaccttttactatgcggaagattaccaccagcagtacctgagcaaggaccccgacgggtactgcggcctcgggggcaccggagtgtcttgtcccctgggtattaaaaagctcgagcagaaactcatctcagaagaggatctggcagcaaatgatatcctggattacaaggatgacgacgataaggtttaaacgcggccgccgacatgcatgtcacttaagtggcaagcttgtagttaattaagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggaaccccactccctctctgcgcgctcgctcgctcactgaggccgggcgaccaaaggtcgcccgacgcccgggctttgcccgggcggcctcagtgagcgagcgagcgcgcagctgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaatccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaaatccattcgccattcaggctgcgcaactgttgggaagggcgatcggtgcgggcctcttcgctattacgccagctggcg

SEQ ID NO: 2 - DNA, 7852 bp, synthetic construct
  misc_feature 1..7852: note = Description of sequence: lentiviral vector
  source 1..7852: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgatactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagctagcgaattcatgctctcggccaccaggagggccctccagctctttcacagcctcttccccatcccgagaatgggtgactccgccgccaagatcgtcagcccccaggaagccttgccgggccggaaggagcccctcgtcgtagcggccaaacatcatgtcaatggcaacagaacagttgaacctttcccagagggaacacagatggctgtatttggaatgggctgtttctggggagctgaaaggaaattctggaccctgaaaggtgtatattcaactcaagttggttttgcaggaggctatactcccaatcctacttataaagaagtctgctcaggaaaaactggccacgcagaagtagtccgggtggtgttccagccagaacacatcagctttgaggaactgctcaaggtcttctgggagaatcatgacccgacccaaggcatgcgccaaggcaatgaccacggctctcagtaccgctcagccatctacccgacctctgccgagcacgtgggggcagccctgaagtccaaggaggactaccagaaggttctgtcggagcatggtttcggcctgatcaccacggacatccgggagggacaaaccttttactatgcggaagattaccaccagcagtacctgagcaaggaccccgacgggtactgcggcctcgggggcaccggagtgtcttgtcccctgggtattaaaaagctcgagcagaaactcatctcagaagaggatctggcagcaaatgatatcctggattacaaggatgacgacgataaggcggccgctgagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaatctaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

(19) United States
(12) Patent Application Publication
(10) Pub. No.: US 2025/0276094 A1
(43) Pub. Date: Sep. 4, 2025

(54) VECTORS EXPRESSING METHIONINE SULFOXIDE REDUCTASE (MSR)

(71) Applicant: University of South Florida, Tampa, FL (US)

(72) Inventor: Manas R. Biswal, Tampa, FL (US)

(21) Appl. No.: 19/068,537

(22) Filed: Mar. 3, 2025

Related U.S. Application Data
(60) Provisional application No. 63/560,036, filed on Mar. 1, 2024.

(51) Int. Cl. A61K 48/00; C12N 9/02; C12N 15/86

(52) U.S. Cl. CPC A61K 48/0058; C12N 9/0051; C12N 15/86; C12Y 108/04011; C12N 2740/15043; C12N 2750/14143

(57) ABSTRACT

Vectors encoding Msr (e.g., MsrA, MsrB1, MsrB2, and/or MsrB3) and methods of treating or preventing retinal tissue damage using same.

### CROSS-REFERENCE TO RELATED APPLICATIONS

This application claims the benefit of priority to U.S. Provisional Application No. 63/560,036, filed Mar. 1, 2024, which is incorporated by reference herein in its entirety.

### REFERENCE TO SEQUENCE LISTING

The sequence listing submitted on Mar. 3, 2025, as an .XML file entitled “11001-217US1_ST26.xml” created on Feb. 28, 2025, and having a file size of 17,480 bytes is hereby incorporated by reference pursuant to 37 C.F.R. § 1.52 (e) (5).

### BACKGROUND

Oxidative stress induced tissue damage is associated with different retinal degenerative diseases causing blindness. The antioxidant enzyme methionine sulfoxide reductase A (MsrA) is reported to support retinal tissue repair. Loss of MsrA function can lead to impaired visual function and potential degeneration of photoreceptor cells. Thus, there is a need to rescue MsrA function to prevent retinal tissue damage. This need and others are at least partially satisfied by the present disclosure.

### SUMMARY

In one aspect, provided is an adeno associated viral vector encoding MsrA and including about 80% similarity or more to SEQ ID NO: 1. In another aspect, provided is a cell including any of the disclosed adeno associated viral vectors.

In yet another aspect, provided is a lentiviral vector encoding MsrA and including about 80% similarity or more to SEQ ID NO: 2. In yet still another aspect, provided is a cell including any of the disclosed lentiviral vectors.

In yet still another aspect, provided is a method of treating or preventing retinal tissue damage in a subject, the method including administering a vector encoding Msr to the subject.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIGS. 1A-1B depict the detection of MsrA (28KD size) in cell lysates using FLAG antibody.

FIGS. 2A-2C depict a WST-1 assay showing increase in cell viability in MsrA overexpressed cells as compared to control cells against Paraquat induced oxidative stress. Significant protection of ARPE cells was observed when stable cells were incubated with Paraquat. Values are reported as average±SD. (***P<0.005 and **P<0.05, n=5).

FIGS. 3A-3C depict a WST-1 assay showing increase in cell viability in MsrA overexpressed cells as compared to control cells against FAC induced oxidative stress. Significant protection of ARPE cells was observed when stable cells were incubated with FAC. Values are reported as average±SD. (***P<0.005 and **P<0.05, n=5).

FIGS. 4A-4B depict western blot analysis of MsrA expression in ARPE-19 and MsrA-overexpressing ARPE-19 cells. Lane 1: Control ARPE-19 cells. Lane 2: ARPE-19 cells transfected with 5 μg MsrA plasmid. Lane 3: ARPE-19 cells transfected with 10 μg MsrA plasmid. Lane 4: ARPE-19 cells transfected with 20 μg MsrA plasmid. The blot demonstrates a dose-dependent increase in MsrA protein expression with increasing plasmid concentration. β-Actin (or appropriate loading control) was used to confirm equal protein loading.

FIG. 5 shows the AAV-bMsrA-Myc-FLAG plasmid design.

FIG. 6 shows the Lenti-bMsrA plasmid design.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of a disease disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, the term “cell” includes progeny. It is also understood that all progenies may not be precisely identical in DNA content, due to deliberate or inadvertent mutations. Variant progeny that have the same function or biological property, as screened for in the originally transformed cell, are included. The “cells” referred to in the present invention generally are prokaryotic or eukaryotic hosts.

As used herein, the term “nucleic acid” or “nucleic acid sequence” refers to the order or sequence of nucleotides along a strand of nucleic acids. In some cases, the order of these nucleotides may determine the order of the amino acids along a corresponding polypeptide chain. The nucleic acid sequence thus codes for the amino acid sequence. The nucleic acid sequence may be single-stranded or double-stranded, as specified, or contain portions of both double-stranded and single-stranded sequences. The nucleic acid sequence may be composed of DNA, both genomic and cDNA, RNA, or a hybrid, where the sequence comprises any combination of deoxyribo- and ribo-nucleotides, and any combination of bases, including uracil (U), adenine (A), thymine (T), cytosine (C), guanine (G), inosine, xanthine hypoxanthine, isocytosine, isoguanine, etc. It may include modified bases, including locked nucleic acids, peptide nucleic acids, and others known to those skilled in the art.

As used herein, “amino acid” refers to a compound containing both amino (—NH2) and carboxyl (—COOH) groups generally separated by one carbon atom. The central carbon atom may contain a substituent that can be either charged, ionizable, hydrophilic, or hydrophobic. Any of 22 basic building blocks of proteins having the formula NH₂—CHR—COOH, where R is different for each specific amino acid, and the stereochemistry is in the ‘L’ configuration. Additionally, the term “amino acid” can optionally include those with an unnatural ‘D’ stereochemistry and modified forms of the ‘D’ and ‘L’ amino acids.

As used herein, “peptide” refers to a chain of amino acids in which each amino acid is connected to the next by a formation of an amide bond. Peptides are generally considered to consist of up to 30 amino acids, or alternatively up to 25 amino acids, or alternatively up to 20 amino acids, or alternatively up to 15 amino acids, or alternatively up to 10 amino acids, or alternatively up to 5 amino acids, or alternatively between about 5-10 amino acids, or alternatively between about 10-15 amino acids, while the term “protein” is applied to compounds containing longer amino acid chains. As used herein, the term “protein domain” refers to a unit of a protein that serves a single role (e.g., functional, structural, etc.). Proteins can include a single domain or multiple domains. As used herein, the term “enzyme” refers to a protein which can catalyze or facilitate a chemical reaction or biological process.

As used herein, the term “gene” refers to a segment of DNA arranged in a linear manner along a chromosome, which codes for a specific protein or segment of protein. A gene typically includes a promoter, a 5′ untranslated region, one or more coding sequences (exons), optionally introns, and a 3′ untranslated region. The gene may further comprise a terminator, enhancers and/or silencers. In some aspects, the gene may be “mutated,” which refers to the replacement, absence, or presence of additional nucleic acids as compared to a control gene. In some aspects, the gene may be “abnormal,” which refers to an atypical presentation of a gene as compared to a control gene. In some aspects, the mutation or abnormality may have a negative effect on the expression of the gene.

### Vectors

In one aspect, provided is an adenoviral vector encoding MsrA and including about 80% similarity or more (e.g., about 81% similarity or more, about 82% similarity or more, about 83% similarity or more, about 84% similarity or more, about 85% similarity or more, about 86% similarity or more, about 87% similarity or more, about 88% similarity or more, about 89% similarity or more, about 90% similarity or more, about 91% similarity or more, about 92% similarity or more, about 93% similarity or more, about 94% similarity or more, about 95% similarity or more, about 96% similarity or more, about 97% similarity or more, about 98% similarity or more, about 99% similarity or more, about 100% similarity) to SEQ ID NO: 1. In some aspects, the adenoviral vector can comprise SEQ ID NO: 1. In some aspects, the adenoviral vector can consist of SEQ ID NO: 1. In another aspect, provided is a cell including any of the disclosed adenoviral vectors. In some aspects, the cell can be mammalian (e.g., human).

In yet another aspect, provided is a lentiviral vector encoding MsrA and including about 80% similarity or more (e.g., about 81% similarity or more, about 82% similarity or more, about 83% similarity or more, about 84% similarity or more, about 85% similarity or more, about 86% similarity or more, about 87% similarity or more, about 88% similarity or more, about 89% similarity or more, about 90% similarity or more, about 91% similarity or more, about 92% similarity or more, about 93% similarity or more, about 94% similarity or more, about 95% similarity or more, about 96% similarity or more, about 97% similarity or more, about 98% similarity or more, about 99% similarity or more, about 100% similarity) to SEQ ID NO: 2. In some aspects, the lentiviral vector can comprise SEQ ID NO: 2. In some aspects, the lentiviral vector can consist of SEQ ID NO: 2. In yet still another aspect, provided is a cell including any of the disclosed lentiviral vectors. In some aspects, the cell can be mammalian (e.g., human).

### Methods

In an aspect, provided is a method of treating or preventing retinal tissue damage in a subject, the method including administering a vector encoding Msr to the subject. In some aspects, the vector can encode MsrA, MsrB1, MsrB2, and/or MsrB3 (e.g., MsrA and MsrB1, MsrA and MsrB2, MsrA and MsrB3, MsrB1 and MsrB2, MsrB1 and MsrB3, MsrB2 and MsrB3, MsrA and MsrB1 and MsrB2, MsrA and MsrB1 and MsrB3, MsrA and MsrB2 and MsrB3, MsrB1 and MsrB2 and MsrB3, or MsrA and MsrB1 and MsrB2 and MsrB3).

As used herein, the term “vector” refers to any moiety which can deliver a nucleic acid sequence into a cell or virus so that the nucleic acid sequence can be replicated and/or expressed by the cell or virus. In some aspects, the vector can be any suitable in vivo gene expression vector. In some aspects, the vector can be a viral vector. For example, in some aspects, the vector can be an adenovirus vector. As used herein, the term “adenovirus,” abbreviated “Ad,” refers to viruses of the adenoviridae family. Adenovirus is a medium-sized (90-100 nm), nonenveloped icosahedral virus containing double-stranded DNA. The term “adenoviridae” refers collectively to adenoviruses of the genera Atadenovirus, Aviadenovirus, Ichtadenovirus, Mastadenovirus, and Siadenovirus. “Adenovirus” includes, but is not limited to human, bovine, ovine, equine, canine, porcine, murine and simian adenovirus species. Human adenoviruses, i.e., adenoviruses that can infect humans, can be classified into subgenera, or species, A-G. Similarly, the term “adenovirus vector” or “adenoviral vector” refers to an adenovirus containing, in the genome thereof, a sequence other than the intrinsic base sequence of the adenovirus, for example any of the disclosed nucleic acids.

In other aspects, the vector can be a retroviral vector. The term “retroviral vector” refers to a vector containing structural and functional genetic elements that are primarily derived from a retrovirus.

In yet other aspects, the vector can be a lentiviral vector. The term “lentiviral vector” refers to a vector including one or more nucleic acid sequences derived from at least a portion of a lentivirus genome.

In some aspects, the vector can be a plasmid. The term “plasmid” refers to an extra chromosomal element often carrying a gene that is not part of the central metabolism of the cell, for example any of the disclosed nucleic acids, and usually in the form of circular double-stranded DNA molecules.

In some aspects, the vector can be a cosmid. In some aspects, the vector can be an artificial chromosome.

In some aspects, the vector can further encode a promoter. In some aspects, the promoter can be small chicken beta-actin, EF1, or a tissue-specific promoter such as a glial cell promoter, neuronal cell promoter, or retinal pigment epithelial cell promoter.

In some aspects, the vector can further encode a detectable signal for quantifying expression of Msr. In some aspects, the detectable signal can be a FLAG epitope sequence, T2A, a fluorescent moiety (e.g., GFP), Myc, or P2A.

In some aspects, the vector can include about 80% similarity or more (e.g., about 81% similarity or more, about 82% similarity or more, about 83% similarity or more, about 84% similarity or more, about 85% similarity or more, about 86% similarity or more, about 87% similarity or more, about 88% similarity or more, about 89% similarity or more, about 90% similarity or more, about 91% similarity or more, about 92% similarity or more, about 93% similarity or more, about 94% similarity or more, about 95% similarity or more, about 96% similarity or more, about 97% similarity or more, about 98% similarity or more, about 99% similarity or more, about 100% similarity) to SEQ ID NO: 1. In some aspects, the vector can comprise SEQ ID NO: 1. In some aspects, the vector can consist of SEQ ID NO: 1. In some aspects, the vector can include any of the disclosed adenoviral vectors.

In some aspects, the vector can include about 80% similarity or more (e.g., about 81% similarity or more, about 82% similarity or more, about 83% similarity or more, about 84% similarity or more, about 85% similarity or more, about 86% similarity or more, about 87% similarity or more, about 88% similarity or more, about 89% similarity or more, about 90% similarity or more, about 91% similarity or more, about 92% similarity or more, about 93% similarity or more, about 94% similarity or more, about 95% similarity or more, about 96% similarity or more, about 97% similarity or more, about 98% similarity or more, about 99% similarity or more, about 100% similarity) to SEQ ID NO: 2. In some aspects, the vector can comprise SEQ ID NO: 2. In some aspects, the vector can consist of SEQ ID NO: 2. In some aspects, the vector can include any of the disclosed lentiviral vectors.

In some aspects, the vector can be encapsulated by a vehicle, and the method can further include delivering said vehicle to the subject. As used herein, the terms “vehicle,” “delivery vehicle,” “transfer vehicle,” “nanoparticle” or grammatical equivalent, are used interchangeably.

In some aspects, the vector may be delivered via a single delivery vehicle. In some aspects, the vector may be delivered via one or more delivery vehicles each of a different composition. According to various aspects, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), cell- or platelet-derived exosomes, ethosomes, or transfersomes.

In some aspects, a suitable delivery vehicle is a lipid nanoparticle. As used herein, “lipid nanoparticles” refer to particles having at least one dimension on the order of nanometers (e.g., 1-1000 nm) and including one or more lipids. In the context of the present invention, a lipid nanoparticle typically serves to transport a desired vector to a target cell or tissue. The process of incorporation of a desired vector into a lipid nanoparticle is often referred to as “loading”. The lipids and the vector can create a self-assembled structure via counterion interactions. The purpose of incorporating a vector into a transfer vehicle, such as a lipid nanoparticle, is often to protect the nucleic acid from an environment which may contain enzymes or chemicals that degrade nucleic acids and/or systems or receptors that cause the rapid excretion of the nucleic acids. Accordingly, in some aspects, a suitable delivery vehicle is capable of enhancing the stability of the vector contained therein and/or facilitate the delivery of vector to the target cell or tissue.

In some aspects, the vector can be transfected into a cell, and wherein the method further comprises delivering said cell to the subject. In some aspects, the cell can be mammalian (e.g., human). In some aspects, the cell can be derived from the subject.

In some aspects, the retinal tissue damage can be caused by oxidative stress.

In some aspects, the vector can be administered in a dose of about 10 μL or more (e.g., about 15 μL or more, about 20 μL or more, about 25 μL or more, about 30 μL or more, about 35 μL or more, about 40 μL or more, about 45 μL or more, about 50 μL or more, about 55 μL or more, about 60 μL or more, about 65 μL or more, about 70 μL or more, about 75 μL or more, about 80 μL or more, about 85 μL or more, about 90 μL or more, about 95 μL or more, about 100 μL or more, about 105 μL or more, about 110 μL or more, about 115 μL or more, about 120 μL or more, about 125 μL or more, about 130 μL or more, about 135 μL or more, about 140 μL or more, about 145 μL or more, about 150 μL or more). In some aspects, the vector can be administered in a dose of about 150 μL or less (e.g., about 145 μL or less, about 140 μL or less, about 135 μL or less, about 130 μL or less, about 125 μL or less, about 120 μL or less, about 115 μL or less, about 110 μL or less, about 105 μL or less, about 100 μL or less, about 95 μL or less, about 90 μL or less, about 85 μL or less, about 80 μL or less, about 75 μL or less, about 70 μL or less, about 65 μL or less, about 60 μL or less, about 55 μL or less, about 50 μL or less, about 45 μL or less, about 40 μL or less, about 35 μL or less, about 30 μL or less, about 25 μL or less, about 20 μL or less, about 15 μL or less, about 10 μL or less). The vector can be administered in a dose ranging from any of the minimum values described above to any of the maximum values described above. For example, in some aspects, the vector can be administered in a dose of from about 10 μL to about 150 μL (e.g., from about 15 μL to about 145 μL, from about 20 μL to about 140 μL, from about 25 μL to about 135 μL, from about 30 μL to about 130 μL, from about 35 μL to about 125 μL, from about 40 μL to about 120 μL, from about 45 μL to about 115 μL, from about 50 μL to about 110 μL, from about 55 μL to about 105 μL, from about 60 μL to about 100 μL, from about 65 μL to about 95 μL, from about 70 μL to about 90 μL, from about 75 μL to about 85 μL, from about 10 μL to about 80 μL, from about 15 μL to about 75 μL, from about 20 μL to about 70 μL, from about 25 μL to about 65 μL, from about 30 μL to about 60 μL, from about 35 μL to about 55 μL, from about 40 μL to about 50 μL, from about 80 μL to about 150 μL, from about 85 μL to about 145 μL, from about 90 μL to about 140 μL, from about 95 μL to about 135 μL, from about 100 μL to about 130 μL, from about 105 μL to about 125 μL, from about 110 μL to about 120 μL).

In some aspects, the vector can be administered once every month, or once every 2 months, or once every 3 months, or once every 4 months, or once every 5 months, or once every 6 months, or once every 7 months, or once every 8 months, or once every 9 months, or once every 10 months, or once every 11 months, or once every year, or once every 1.5 years, or once every 2 years, or once every 2.5 years, or once every 3 years, or once every 3.5 years, or once every 4 years, or once every 4.5 years, or once every 5 years.

In some aspects, the vector can be administered intraocularly or via eye drops. In some aspects, the vector can be administered into the retina through intravitreal, subretinal, or suprachoroidal injection.

In some aspects, the vector can be administered to retinal pigment epithelium (RPE) of the subject.

### EXAMPLES

### Example 1

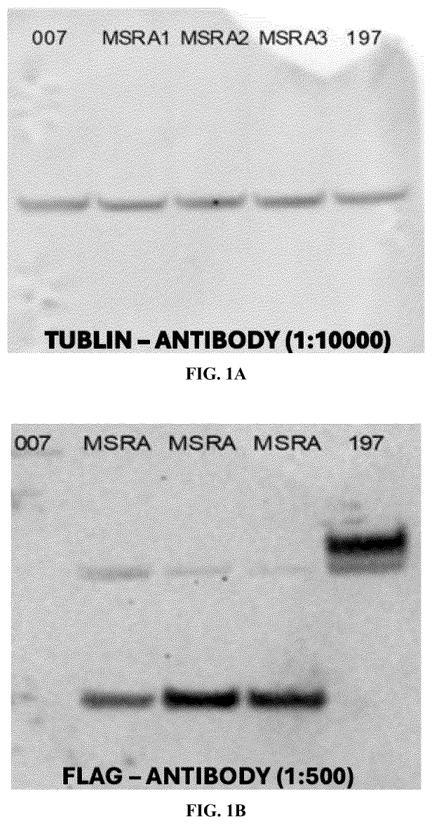

Oxidative stress induced tissue damage is associated with different retinal degenerative diseases causing blindness. The antioxidant enzyme methionine sulfoxide reductase A (MsrA) is reported to support retinal tissue repair. A study was conducted to develop an Adeno Associated vector (AAV) to express MsrA so that it can be exploited in tissue repair under oxidative damage. Human MsrA gene was cloned into self-complementary AAV (scAAV) plasmid vector under a ubiquitous small chicken beta-actin (smCBA) promoter. Further to track exogenous expression, a FLAG epitope sequence was further cloned into the 3′ end replacing the stop codon. As a comparison, a control plasmid driving green fluorescent protein (GFP) was also developed. These plasmids were transfected into HELA cells (1.5×10⁵ cell/well in a six well plate) using polyethyleneimine (PEI) as a transfection reagent and cell lysates were analyzed by western blotting. The results showed efficient expression of GFP fluorescence suggesting 70% efficiency of plasmid transfection. 20 μg proteins isolated from cellular lysates (three replicates) were analyzed by western blotting, and use of FLAG antibody showed MsrA protein expression of 28KD size (FIGS. 1A-1B). The designed scAAV-MsrA plasmid vector can be used in cellular and animal models of retinal degenerations.

### Example 2

MsrA plays a crucial role in the eye by protecting retinal cells from oxidative stress through repairing oxidized methionine residues on proteins, essentially acting as an antioxidant, particularly important in the retinal pigment epithelium (RPE) where high levels of oxidative stress can occur; loss of MSRA function can lead to impaired visual function and potential degeneration of photoreceptor cells. A study was conducted which hypothesized that presence of Methionine Sulfoxide Reductase A (MsrA) can protect RPE cells from oxidative stress-induced cell death.

hMsrA cDNA was PCR-amplified and cloned into a lentiviral plasmid (pCDH backbone) under the EF1 promoter with a T2A tag. The plasmids were prepared using maxiprep and transfected into HeLa cells to confirm the expression of T2A and β-Actin. Lentivirus production was performed by triple transfection in HEK-293T cells, followed by purification using puromycin-mediated selection. The purified virus was successfully transduced into ARPE-19 cells, and stable expression was verified through PCR and protein analyses.

ARPE-19 and MsrA stable ARPE19 cells were seeded in 96-well plates at a density of 6000 to 10000 cells per well and allowed to adhere overnight. Following adherence, cells were treated with varying concentrations of paraquat and FAC for a specified duration. (The media was replaced with 150 μl of DMEM-F12 serum-free media containing 250 and 500 μM of Paraquat and FAC for 24 hours). Control wells received equivalent volumes of the vehicle solution. After treatment, 100 μL DMEM-F12 serum-free media containing 10 μL of WST-1 reagent was added to each well, and the plates were incubated for an additional two hours at 37° C. Absorbance was measured at 450 nm using a microplate reader. Data were analyzed using GraphPad-Prism (version 9), and viability percentages were calculated relative to vehicle-treated controls. All experiments were conducted in triplicate and repeated at least three times to ensure statistical significance.

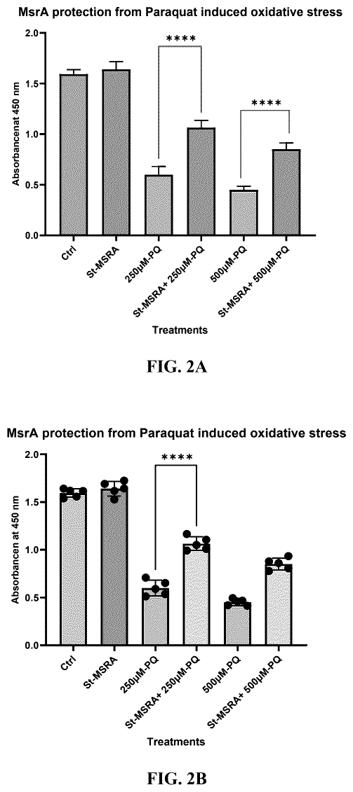

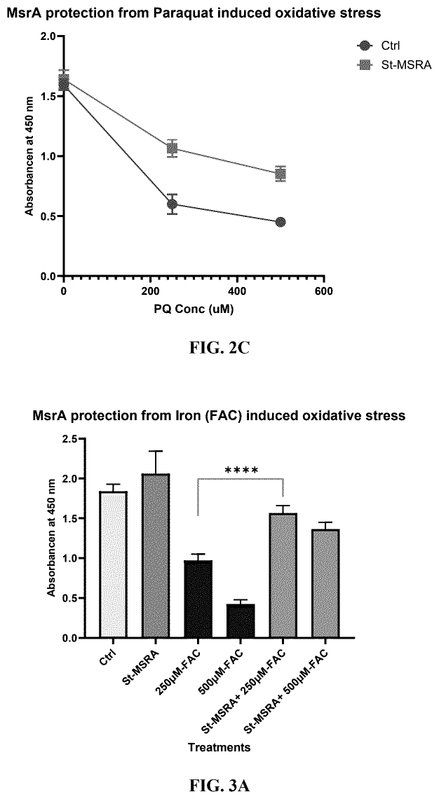

FIGS. 2A-2C depict a WST-1 assay showing increase in cell viability upon lenti-MsrA expression as compared to control cells against Paraquat induced oxidative stress. Significant protection of ARPE cells was observed when stable cells were incubated with 250 and 500 μM of Paraquat. Values are reported as average±SD. (***P<0.005 and **P<0.05, n=5).

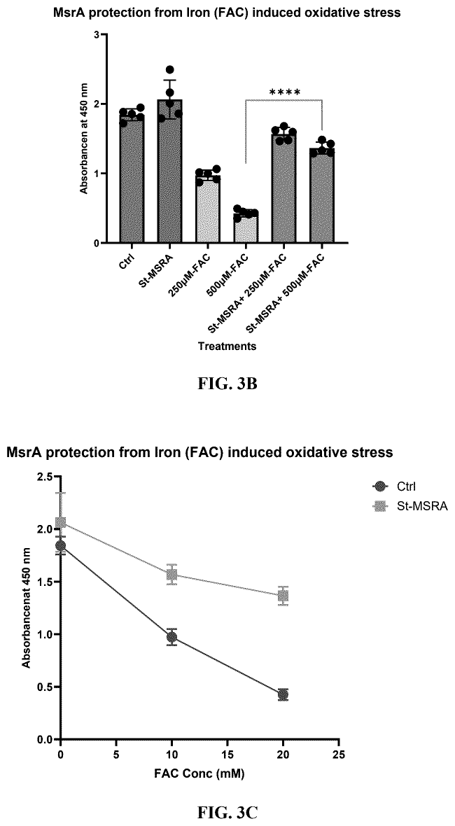

FIGS. 3A-3C depict a WST-1 assay showing increase in cell viability upon lenti-MsrA expression as compared to control cells against FAC induced oxidative stress. Significant protection of ARPE cells was observed when stable cells were incubated with 250 and 500 M of FAC. Values are reported as average±SD. (***P<0.005 and **P<0.05, n=5).

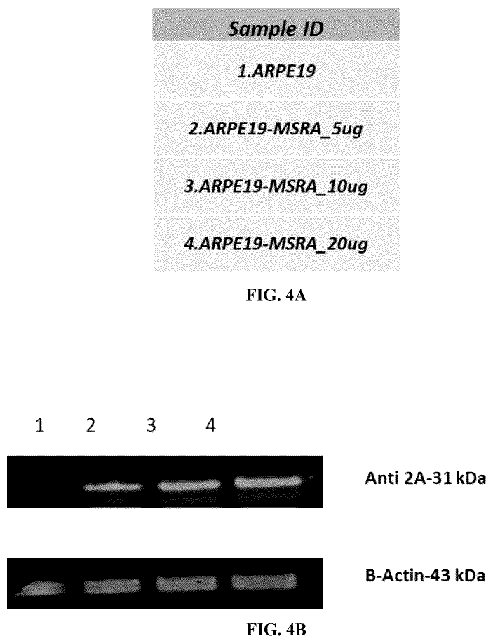

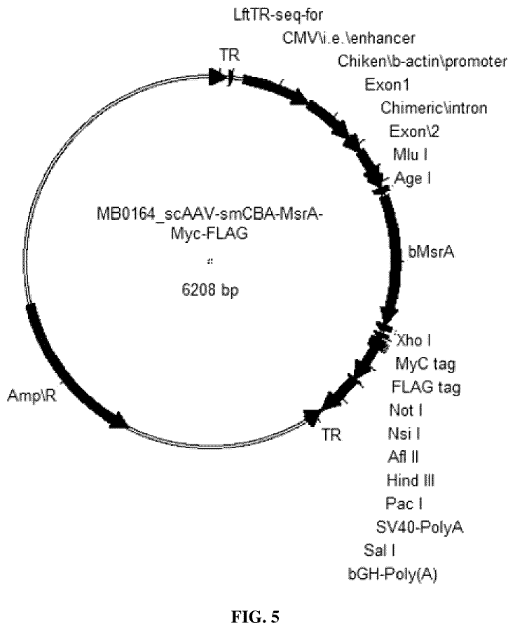

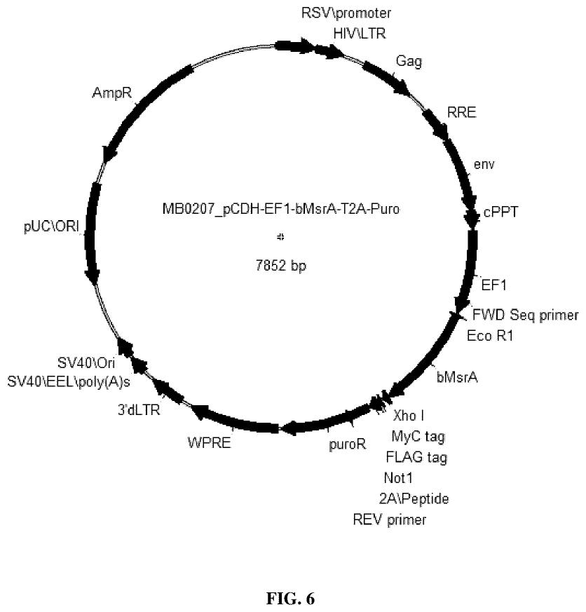

FIGS. 4A-4B depict detection of MsrA in stably transfected ARPE-19 cells and further validation of plasmid by antibodies against β-actin and Anti2A tag.

### Example 3

Methionine sulfoxide reductases (Msrs) exist in multiple variants, each with distinct substrate specificity and cellular functions. MsrA reduces the S-epimer of methionine sulfoxide (Met-SO) back to methionine and plays a role in antioxidant defense, protein repair, and neuroprotection, residing in the cytosol, mitochondria, and nucleus. MsrB specifically reduces the R-epimer of methionine sulfoxide (Met-RO) and exists in three isoforms: MsrB1 (cytoplasm and nucleus), MsrB2 (mitochondria), and MsrB3 (endoplasmic reticulum and mitochondria). MsrC, though less studied, is found in some bacterial and plant species and functions similarly to MsrA but has different evolutionary origins. The primary difference between MsrA and MsrB is their substrate specificity, targeting different epimers of methionine sulfoxide. The Msr system collectively helps counteract oxidative damage and maintain cellular longevity and function.

Methionine Sulfoxide Reductase A (MsrA) is an essential enzyme that plays a critical role in protecting cells from oxidative stress by reducing methionine sulfoxide back to methionine. This enzymatic repair mechanism is vital for maintaining protein function and cellular homeostasis, particularly in tissues exposed to high oxidative stress, such as the retina. MsrA functions as a key component of the cellular antioxidant defense system by reversing the oxidation of methionine residues in proteins, which occurs due to reactive oxygen species (ROS). This function is crucial for preserving protein structure and function, thereby preventing age-related and disease-associated damage. MsrA has been implicated in multiple physiological and pathological processes, including neuroprotection, aging, and retinal degenerative diseases.

In the context of retinal health, MsrA expression has been observed in the retinal pigment epithelium (RPE) and photoreceptor cells, where it helps mitigate oxidative damage. Studies suggest that a deficiency in MsrA leads to increased susceptibility to oxidative stress and exacerbates retinal degeneration. Enhancing MsrA activity through gene therapy or pharmacological approaches presents a promising avenue for developing novel therapeutics targeting age-related macular degeneration (AMD) and other retinal diseases. Recent research efforts have focused on the potential of MsrA-based therapies for mitigating mitochondrial dysfunction, which is a significant contributor to retinal degenerative conditions. The integration of MsrA within an adeno-associated virus (AAV) delivery system for targeted expression in retinal cells holds potential for protecting against oxidative stress-induced damage and preserving visual function. Overall, MsrA serves as a critical antioxidant enzyme with promising therapeutic potential. Understanding its mechanisms and developing effective strategies to enhance its function could significantly contribute to the advancement of treatments for oxidative stress-related retinal diseases.

To investigate the protective role of Methionine Sulfoxide Reductase A (MsrA) in retinal pigment epithelium (RPE) cells, a study was conducted which designed cloning strategies for both Adeno-Associated Virus (AAV) and Lentiviral (Lenti) plasmids.

Cloning MsrA into AAV Plasmid:

Vector Selection: The study selected an AAV expression plasmid containing a strong promoter small chicken beta actin (smCBA) to drive MsrA expression in RPE cells. Bovine MsrA cDNA was amplified using PCR with primers (NCBI Sequence ID: BC102980.1)) incorporating restriction enzyme sites compatible with the multiple cloning site (MCS) of the AAV plasmid. The PCR product and AAV plasmid were digested with selected restriction enzymes and ligated using T4 DNA ligase. The ligated plasmid was transformed into competent E. coli cells, and colonies were screened via colony PCR and sequencing. The validated plasmid was packaged into AAV particles using a helper-free system in HEK293T cells.

Cloning MsrA into Lenti Plasmid: A lentiviral vector (pCDH) with an EFla promoter was chosen for stable MsrA expression. Bovine MsrA cDNA was amplified with primers containing restriction sites or Gateway recombination sequences. The insert was ligated into the lentiviral backbone via restriction digestion-ligation or recombination cloning. Recombinant plasmids were transformed into E. coli, and positive clones were verified through restriction digestion and sequencing. The validated construct was co-transfected with packaging plasmids into HEK293T cells, and viral supernatant was collected and concentrated for RPE transduction. Transduced RPE cells were selected using antibiotics to generate stable cell lines expressing MsrA.

Validation of MsrA expression in Stable ARPE-19 cells: Proteins isolated from control and stable MsrA ARPE-19 cells were analyzed by western blotting. To detect exogenous MsrA expression, the study used the T2A antibody (Tagged to MsrA) and β-actin antibody was used as a control.

MsrA provided cellular protection from Paraquat and Ferric ammonium citrate (FAC) induced oxidative stress in ARPE19: Control and MsrA-stable ARPE-19 cells were seeded in 96-well plates (6,000-10,000 cells/well) and allowed to adhere overnight. Cells were then treated with 250 μM and 500 μM paraquat and FAC in serum-free DMEM-F12 for 24 hours to induce oxidative stress and ferroptosis-mediated cell death, while control wells received vehicle-only treatment. Following incubation, WST-1 reagent was added, and absorbance was measured at 450 nm to assess cell viability. Data were analyzed using GraphPad Prism (version 9), with viability calculated relative to controls. All experiments were performed in triplicate and repeated at least three times to ensure statistical reliability.

Any patents, applications and publications as listed below and throughout this document are hereby incorporated by reference in their entirety herein.

SEQUENCES

SEQ ID NO: 1 (AAV vector)
cgctcgctcgctcactgaggccgcccgggcaaagcccgggcgtcgggcgacctttggtcgcccggcctcagtgagcgagcgagcgcg
cagagagggagtggccaactccatcactaggggttccttgtagttaatgattaacccgccatgctacttatctacgtagccatgctcgatctga
attcggtaccctagttattaatagtaatcaattacggggtcattagttcatagcccatatatggagttccgcgttacataacttacggtaaatggcc
cgcctggctgaccgcccaacgacccccgcccattgacgtcaataatgacgtatgttcccatagtaacgccaatagggactttccattgacgt
caatgggtggactatttacggtaaactgcccacttggcagtacatcaagtgtatcatatgccaagtacgccccctattgacgtcaatgacggta
aatggcccgcctggcattatgcccagtacatgaccttatgggactttcctacttggcagtacatctacgtattagtcatcgctattaccatggtcg
aggtgagccccacgttctgcttcactctccccatctcccccccctccccacccccaattttgtatttatttattttttaattattttgtgcagcgatgg
gggcggggggggggggggggcgcgcgccaggcggggggggcggggcgaggggggggggggcgaggcggagaggtgcg
gcggcagccaatcagagcggcgcgctccgaaagtttccttttatggcgaggcggcggcggcggcggccctataaaaagcgaagcgcgc
ggcggggggagtcgctgcgacgctgccttcgccccgtgccccgctccgccgccgcctcgcgccgcccgccccggctctgactgaccg
cgttactcccacaggtgagcggggggacggcccttctcctccgggctgtaattagcgcttggtttaatgacggcttgtttcttttctgtggctg
cgtgaaagccttgaggggctccgggagctagagcctctgctaaccatgttcatgccttcttctttttcctacagctcctgggcaacgtgctggtt
attgtgctgtctcatcattttggcaaagaattcctcgaagatctaggcaacgcgtTGGATCCACCGGTGAGGAGATCTG
CCGCCGCGATCGCCATGCTCTCGGCCACCAGGAGGGCCCTCCAGCTCTTTCACAGCC
TCTTCCCCATCCCGAGAATGGGTGACTCCGCCGCCAAGATCGTCAGCCCCCAGGAAG
CCTTGCCGGGCCGGAAGGAGCCCCTCGTCGTAGCGGCCAAACATCATGTCAATGGC
AACAGAACAGTTGAACCTTTCCCAGAGGGAACACAGATGGCTGTATTTGGAATGGG
CTGTTTCTGGGGAGCTGAAAGGAAATTCTGGACCCTGAAAGGTGTATATTCAACTCA
AGTTGGTTTTGCAGGAGGCTATACTCCCAATCCTACTTATAAAGAAGTCTGCTCAGG
AAAAACTGGCCACGCAGAAGTAGTCCGGGTGGTGTTCCAGCCAGAACACATCAGCT
TTGAGGAACTGCTCAAGGTCTTCTGGGAGAATCATGACCCGACCCAAGGCATGCGC
CAAGGCAATGACCACGGCTCTCAGTACCGCTCAGCCATCTACCCGACCTCTGCCGAG
CACGTGGGGGCAGCCCTGAAGTCCAAGGAGGACTACCAGAAGGTTCTGTCGGAGCA
TGGTTTCGGCCTGATCACCACGGACATCCGGGAGGGACAAACCTTTTACTATGCGGA
AGATTACCACCAGCAGTACCTGAGCAAGGACCCCGACGGGTACTGCGGCCTCGGGG
GCACCGGAGTGTCTTGTCCCCTGGGTATTAAAAAGCTCGAGCAGAAACTCATCTCAG
AAGAGGATCTGGCAGCAAATGATATCCTGGATTACAAGGATGACGACGATAAGGTT
TAAACGCGGCCGCCGACATGCATGTCACTTAAGTGGCAAGCTTGTAGTTAATTAAGA
TCCAGACATGATAAGATACATTGATGAGTTTGGACAAACCACAACTAGAATGCAGT
GAAAAAAATGCTTTATTTGTGAAATTTGTGATGCTATTGCTTTATTTGTAACCATTAT
AAGCTGCAATAAACAAGTTAACAACAACAATTGCATTCATTTTATGTTTCAGGTTCA
GGGGGAGGTGTGGGAGGTTTTTTAGTCGACtagagctcgctgatcagcctcgactgtgccttctagttgccagc
catctgttgtttgcccctcccccgtgccttccttgaccctggaaggtgccactcccactgtcctttcctaataaaatgaggaaattgcatcgcatt
gtctgagtaggtgtcattctattctggggggggggtggggcaggacagcaagggggaggattgggaagacaatagcaggaaccccact
ccctctctgcgcgctcgctcgctcactgaggccgggcgaccaaaggtcgcccgacgcccgggctttgcccgggcggcctcagtgagcg
agcgagcgcgcagctgctgcattaatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggttatccacagaatcaggggataac
gcaggaaagaacatgtgagcaaaaggccagcaaaaggccaggaaccgtaaaaaggccgcgttgctggcgtttttccataggctccgccc
ccctgacgagcatcacaaaaatcgacgctcaagtcagaggtggcgaaacccgacaggactataaagataccaggcgtttccccctggaa
gctccctcgtgcgctctcctgttccgaccctgccgcttaccggatacctgtccgcctttctcccttcgggaagcgtggcgctttctcatagctca
cgctgtaggtatctcagttcggtgtaggtcgttcgctccaagctgggctgtgtgcacgaaccccccgttcagcccgaccgctgcgccttatcc
ggtaactatcgtcttgagtccaacccggtaagacacgacttatcgccactggcagcagccactggtaacaggattagcagagcgaggtatg
taggcggtgctacagagttcttgaagtggtggcctaactacggctacactagaaggacagtatttggtatctgcgctctgctgaagccagtta
ccttcggaaaaagagttggtagctcttgatccggcaaacaaaccaccgctggtagcggtggtttttttgtttgcaagcagcagattacgcgca
gaaaaaaaggatctcaagaagatcctttgatcttttctacggggtctgacgctcagtggaacgaaaactcacgttaagggattttggtcatgag
attatcaaaaaggatcttcacctagatccttttaaattaaaaatgaagttttaaatcaatctaaagtatatatgagtaaacttggtctgacagttacc
aatgcttaatcagtgaggcacctatctcagcgatctgtctatttcgttcatccatagttgcctgactccccgtcgtgtagataactacgatacggg
agggcttaccatctggccccagtgctgcaatgataccgcgagacccacgctcaccggctccagatttatcagcaataaaccagccagccg
gaagggccgagcgcagaagtggtcctgcaactttatccgcctccatccagtctattaattgttgccgggaagctagagtaagtagttcgcca
gttaatagtttgcgcaacgttgttgccattgctacaggcatcgtggtgtcacgctcgtcgtttggtatggcttcattcagctccggttcccaacga
tcaaggcgagttacatgatcccccatgttgtgcaaaaaagcggttagctccttcggtcctccgatcgttgtcagaagtaagttggccgcagtgt
tatcactcatggttatggcagcactgcataattctcttactgtcatgccatccgtaagatgcttttctgtgactggtgagtactcaaccaagtcatt
ctgagaatagtgtatgcggcgaccgagttgctcttgcccggcgtcaatacgggataataccgcgccacatagcagaactttaaaagtgctca
tcattggaaaacgttcttcggggcgaaaactctcaaggatcttaccgctgttgagatccagttcgatgtaacccactcgtgcacccaactgatc
ttcagcatcttttactttcaccagcgtttctgggtgagcaaaaacaggaaggcaaaatgccgcaaaaaagggaataagggcgacacggaaa
tgttgaatactcatactcttcctttttcaatattattgaagcatttatcagggttattgtctcatgagcggatacatatttgaatgtatttagaaaaataa
acaaataggggttccgcgcacatttccccgaaaagtgccacctgacgtctaagaaaccattattatcatgacattaacctataaaaataggcgt
atcacgaggccctttcgtctcgcgcgtttcggtgatgacggtgaaaacctctgacacatgcagctcccggagacggtcacagcttgtctgta
agcggatgccgggagcagacaagcccgtcagggcgcgtcagcgggtgttggcgggtgtcggggctggcttaactatgcggcatcagag
cagattgtactgagagtgcaccatatgcggtgtgaaataccgcacagatgcgtaaggagaaaataccgcatcaggaaatccaacatccaat
aaatcatacaggcaaggcaaagaattagcaaaattaagcaataaagcctcagagcataaagctaaatcggttgtaccaaaaacattatgacc
ctgtaatacttttgcgggagaagcctttatttcaacgcaaggataaaaatttttagaaccctcatatattttaaatgcaatgcctgagtaatgtgta
ggtaaagattcaaacgggtgagaaaggccggagacagtcaaatcaccatcaatatgatattcaaccgttctagctgataaattcatgccgga
gagggtagctatttttgagaggtctctacaaaggctatcaggtcattgcctgagagtctggagcaaacaagagaatcgatgaacggtaatcgt
aaaactagcatgtcaatcatatgtaccccggttgataatcagaaaagccccaaaaacaggaagattgtataagcaaatatttaaattgtaaacg
ttaatattttgttaaaattcgcgttaaatttttgttaaatcagctcattttttaaccaataggccgaaatcggcaaaatcccttataaatcaaaagaat
agaccgagatagggttgagtgttgttccagtttggaacaagagtccactattaaagaacgtggactccaacgtcaaagggcgaaaaaccgt
ctatcagggcgatggcccactacgtgaaccatcaccctaatcaagttttttggggtcgaggtgccgtaaagcactaaatcggaaccctaaag
ggagcccccgatttagagcttgacggggaaagccggcgaacgtggcgagaaaggaagggaagaaagcgaaaggagcgggcgctag
ggcgctggcaagtgtagcggtcacgctgcgcgtaaccaccacacccgccgcgcttaatgcgccgctacagggcgcgtactatggttgcttt
gacgagcacgtataacgtgctttcctcgttagaatcagagcgggagctaaacaggaggccgattaaagggattttagacaggaacggtacg
ccagaatcctgagaagtgtttttataatcagtgaggccaccgagtaaaagagtctgtccatcacgcaaattaaccgttgtcgcaatacttctttg
attagtaataacatcacttgcctgagtagaagaactcaaactatcggccttgctggtaatatccagaacaatattaccgccagccattgcaaca
ggaaaaacgctcatggaaatacctacattttgacgctcaatcgtctggaaatccattcgccattcaggctgcgcaactgttgggaagggcgat
cggtgcgggcctcttcgctattacgccagctggcg
SEQ ID NO: 2 (lentiviral vector)
acgcgtgtagtcttatgcaatactcttgtagtcttgcaacatggtaacgatgagttagcaacatgccttacaaggagagaaaaagcaccgtgc
atgccgattggtggaagtaaggtggtacgatcgtgccttattaggaaggcaacagacgggtctgacatggattggacgaaccactgaattg
ccgcattgcagagatattgtatttaagtgcctagctcgatacaataaacgggtctctctggttagaccagatctgagcctgggagctctctggc
taactagggaacccactgcttaagcctcaataaagcttgccttgagtgcttcaagtagtgtgtgcccgtctgttgtgtgactctggtaactagag
atccctcagacccttttagtcagtgtggaaaatctctagcagtggcgcccgaacagggacctgaaagcgaaagggaaaccagagctctctc
gacgcaggactcggcttgctgaagcgcgcacggcaagaggcgaggggcggcgactggtgagtacgccaaaaattttgactagcggag
gctagaaggagagagatgggtgcgagagcgtcagtattaagcgggggagaattagatcgcgatgggaaaaaattcggttaaggccagg
gggaaagaaaaaatataaattaaaacatatagtatgggcaagcagggagctagaacgattcgcagttaatcctggcctgttagaaacatcag
aaggctgtagacaaatactgggacagctacaaccatcccttcagacaggatcagaagaacttagatcattatataatacagtagcaaccctct
attgtgtgcatcaaaggatagagataaaagacaccaaggaagctttagacaagatagaggaagagcaaaacaaaagtaagaccaccgca
cagcaagcggccactgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttccttggg
ttcttgggagcagcaggaagcactatgggcgcagcctcaatgacgctgacggtacaggccagacaattattgtctggtatagtgcagcagc
agaacaatttgctgagggctattgaggcgcaacagcatctgttgcaactcacagtctggggcatcaagcagctccaggcaagaatcctggc
tgtggaaagatacctaaaggatcaacagctcctggggatttggggttgctctggaaaactcatttgcaccactgctgtgccttggaatgctagt
tggagtaataaatctctggaacagattggaatcacacgacctggatggagtgggacagagaaattaacaattacacaagcttaatacactcct
taattgaagaatcgcaaaaccagcaagaaaagaatgaacaagaattattggaattagataaatgggcaagtttgtggaattggtttaacataa
caaattggctgtggtatataaaattattcataatgatagtaggaggcttggtaggtttaagaatagtttttgctgtactttctatagtgaatagagtta
ggcagggatattcaccattatcgtttcagacccacctcccaaccccgaggggacccgacaggcccgaaggaatagaagaagaaggtgga
gagagagacagagacagatccattcgattagtgaacggatctcgacggttaacttttaaaagaaaaggggggattggggggtacagtgca
ggggaaagaatagtagacataatagcaacagacatacaaactaaagaattacaaaaacaaattacaaaaattcaaaattttatcgatactagt
aaggatctgcgatcgctccggtgcccgtcagtgggcagagcgcacatcgcccacagtccccgagaagttggggggaggggtcggcaat
tgaacgggtgcctagagaaggtggcgcggggtaaactgggaaagtgatgtcgtgtactggctccgcctttttcccgaggggggggagaa
ccgtatataagtgcagtagtcgccgtgaacgttctttttcgcaacgggtttgccgccagaacacagctgaagcttcgaggggctcgcatctct
ccttcacgcgcccgccgccctacctgaggccgccatccacgccggttgagtcgcgttctgccgcctcccgcctgtggtgcctcctgaactg
cgtccgccgtctaggtaagtttaaagctcaggtcgagaccgggcctttgtccggcgctcccttggagcctacctagactcagccggctctcc
acgctttgcctgaccctgcttgctcaactctacgtctttgtttcgttttctgttctgcgccgttacagatccaagctgtgaccggcgcctacTCT
AGAGCTAGCGAATTCATGCTCTCGGCCACCAGGAGGGCCCTCCAGCTCTTTCACAGC
CTCTTCCCCATCCCGAGAATGGGTGACTCCGCCGCCAAGATCGTCAGCCCCCAGGAA
GCCTTGCCGGGCCGGAAGGAGCCCCTCGTCGTAGCGGCCAAACATCATGTCAATGG
CAACAGAACAGTTGAACCTTTCCCAGAGGGAACACAGATGGCTGTATTTGGAATGG
GCTGTTTCTGGGGAGCTGAAAGGAAATTCTGGACCCTGAAAGGTGTATATTCAACTC
AAGTTGGTTTTGCAGGAGGCTATACTCCCAATCCTACTTATAAAGAAGTCTGCTCAG
GAAAAACTGGCCACGCAGAAGTAGTCCGGGTGGTGTTCCAGCCAGAACACATCAGC
TTTGAGGAACTGCTCAAGGTCTTCTGGGAGAATCATGACCCGACCCAAGGCATGCGC
CAAGGCAATGACCACGGCTCTCAGTACCGCTCAGCCATCTACCCGACCTCTGCCGAG
CACGTGGGGGCAGCCCTGAAGTCCAAGGAGGACTACCAGAAGGTTCTGTCGGAGCA
TGGTTTCGGCCTGATCACCACGGACATCCGGGAGGGACAAACCTTTTACTATGCGGA
AGATTACCACCAGCAGTACCTGAGCAAGGACCCCGACGGGTACTGCGGCCTCGGGG
GCACCGGAGTGTCTTGTCCCCTGGGTATTAAAAAGCTCGAGCAGAAACTCATCTCAG
AAGAGGATCTGGCAGCAAATGATATCCTGGATTACAAGGATGACGACGATAAGGCG
GCCGCtgagggcagaggaagtcttctaacatgcggtgacgtggaggagaatcccggcccttccgggatgaccgagtacaagcccac
ggtgcgcctcgccacccgcgacgacgtccccagggccgtacgcaccctcgccgccgcgttcgccgactaccccgccacgcgccacac
cgtcgatccggaccgccacatcgagcgggtcaccgagctgcaagaactcttcctcacgcgcgtcgggctcgacatcggcaaggtgtggg
tcgcggacgacggcgccgcggtggcggtctggaccacgccggagagcgtcgaagcgggggcggtgttcgccgagatcggcccgcgc
atggccgagttgagcggttcccggctggccgcgcagcaacagatggaaggcctcctggcgccgcaccggcccaaggagcccgcgtgg
ttcctggccaccgtcggcgtctcgcccgaccaccagggcaagggtctgggcagcgccgtcgtgctccccggagtggaggcggccgagc
gcgccggggtgcccgccttcctggagacctccgcgccccgcaacctccccttctacgagcggctcggcttcaccgtcaccgccgacgtc
gaggtgcccgaaggaccgcgcacctggtgcatgacccgcaagcccggtgcctgaatctaggtcgacaatcaacctctggattacaaaattt
gtgaaagattgactggtattcttaactatgttgctccttttacgctatgtggatacgctgctttaatgcctttgtatcatgctattgcttcccgtatggc
tttcattttctcctccttgtataaatcctggttgctgtctctttatgaggagttgtggcccgttgtcaggcaacgtggcgtggtgtgcactgtgtttgc
tgacgcaacccccactggttggggcattgccaccacctgtcagctcctttccgggactttcgctttccccctccctattgccacggcggaact
catcgccgcctgccttgcccgctgctggacaggggctcggctgttgggcactgacaattccgtggtgttgtcggggaaatcatcgtcctttcc
ttggctgctcgcctgtgttgccacctggattctgcgcgggacgtccttctgctacgtcccttcggccctcaatccagcggaccttccttcccgc
ggcctgctgccggctctgcggcctcttccgcgtcttcgccttcgccctcagacgagtcggatctccctttgggccgcctccccgcctggtac
ctttaagaccaatgacttacaaggcagctgtagatcttagccactttttaaaagaaaaggggggactggaagggctaattcactcccaacgaa
aataagatctgctttttgcttgtactgggtctctctggttagaccagatctgagcctgggagctctctggctaactagggaacccactgcttaag
cctcaataaagcttgccttgagtgcttcaagtagtgtgtgcccgtctgttgtgtgactctggtaactagagatccctcagacccttttagtcagtg
tggaaaatctctagcagtagtagttcatgtcatcttattattcagtatttataacttgcaaagaaatgaatatcagagagtgagaggaacttgtttat
tgcagcttataatggttacaaataaagcaatagcatcacaaatttcacaaataaagcatttttttcactgcattctagttgtggtttgtccaaactca
tcaatgtatcttatcatgtctggctctagctatcccgcccctaactccgcccagttccgcccattctccgccccatggctgactaattttttttattta
tgcagaggccgaggccgcctcggcctctgagctattccagaagtagtgaggaggcttttttggaggcctagacttttgcagagacggccca
aattcgtaatcatggtcatagctgtttcctgtgtgaaattgttatccgctcacaattccacacaacatacgagccggaagcataaagtgtaaagc
ctggggtgcctaatgagtgagctaactcacattaattgcgttgcgctcactgcccgctttccagtcgggaaacctgtcgtgccagctgcattaa
tgaatcggccaacgcgcggggagaggcggtttgcgtattgggcgctcttccgcttcctcgctcactgactcgctgcgctcggtcgttcggct
gcggcgagcggtatcagctcactcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccctgacgagcatcacaaaaatcgac
gctcaagtcagaggtggcgaaacccgacaggactataaagataccaggcgtttccccctggaagctccctcgtgcgctctcctgttccgac
cctgccgcttaccggatacctgtccgcctttctcccttcgggaagcgtggcgctttctcatagctcacgctgtaggtatctcagttcggtgtagg
tcgttcgctccaagctgggctgtgtgcacgaaccccccgttcagcccgaccgctgcgccttatccggtaactatcgtcttgagtccaacccg
gtaagacacgacttatcgccactggcagcagccactggtaacaggattagcagagcgaggtatgtaggcggtgctacagagttcttgaagt
ggtggcctaactacggctacactagaaggacagtatttggtatctgcgctctgctgaagccagttaccttcggaaaaagagttggtagctctt
gatccggcaaacaaaccaccgctggtagcggtggtttttttgtttgcaagcagcagattacgcgcagaaaaaaaggatctcaagaagatcct
ttgatcttttctacggggtctgacgctcagtggaacgaaaactcacgttaagggattttggtcatgagattatcaaaaaggatcttcacctagat
ccttttaaattaaaaatgaagttttaaatcaatctaaagtatatatgagtaaacttggtctgacagttaccaatgcttaatcagtgaggcacctatct
cagcgatctgtctatttcgttcatccatagttgcctgactccccgtcgtgtagataactacgatacgggagggcttaccatctggccccagtgct
gcaatgataccgcgagacccacgctcaccggctccagatttatcagcaataaaccagccagccggaagggccgagcgcagaagtggtc
ctgcaactttatccgcctccatccagtctattaattgttgccgggaagctagagtaagtagttcgccagttaatagtttgcgcaacgttgttgcca
ttgctacaggcatcgtggtgtcacgctcgtcgtttggtatggcttcattcagctccggttcccaacgatcaaggcgagttacatgatcccccat
gttgtgcaaaaaagcggttagctccttcggtcctccgatcgttgtcagaagtaagttggccgcagtgttatcactcatggttatggcagcactg
cataattctcttactgtcatgccatccgtaagatgcttttctgtgactggtgagtactcaaccaagtcattctgagaatagtgtatgcggcgaccg
agttgctcttgcccggcgtcaatacgggataataccgcgccacatagcagaactttaaaagtgctcatcattggaaaacgttcttcggggcga
aaactctcaaggatcttaccgctgttgagatccagttcgatgtaacccactcgtgcacccaactgatcttcagcatcttttactttcaccagcgttt
ctgggtgagcaaaaacaggaaggcaaaatgccgcaaaaaagggaataagggcgacacggaaatgttgaatactcatactcttcctttttca
atattattgaagcatttatcagggttattgtctcatgagcggatacatatttgaatgtatttagaaaaataaacaaataggggttccgcgcacattt
ccccgaaaagtgccacctgacgtctaagaaaccattattatcatgacattaacctataaaaataggcgtatcacgaggccctttcgtctcgcgc
gtttcggtgatgacggtgaaaacctctgacacatgcagctcccggagacggtcacagcttgtctgtaagcggatgccgggagcagacaag
cccgtcagggcgcgtcagcgggtgttggcgggtgtcggggctggcttaactatgcggcatcagagcagattgtactgagagtgcaccatat
gcggtgtgaaataccgcacagatgcgtaaggagaaaataccgcatcaggcgccattcgccattcaggctgcgcaactgttgggaagggc
gatcggtgcgggcctcttcgctattacgccagctggcgaaagggggatgtgctgcaaggcgattaagttgggtaacgccagggttttccca
gtcacgacgttgtaaaacgacggccagtgccaagctg

What is claimed is:

1. An adenoviral vector encoding MsrA and comprising about 80% similarity or more to SEQ ID NO: 1.

2. A cell comprising the adenoviral vector of claim 1.

3. A lentiviral vector encoding MsrA and comprising about 80% similarity or more to SEQ ID NO: 2.

4. A cell comprising the lentiviral vector of claim 3.

5. A method of treating or preventing retinal tissue damage in a subject, the method comprising administering a vector encoding Msr to the subject.

6. The method of claim 5, wherein the vector encodes MsrA, MsrB1, MsrB2, and/or MsrB3.

7. The method of claim 5, wherein the vector is an adenoviral vector, a lentiviral vector, or a retroviral vector.

8. The method of claim 5, wherein the vector further encodes a promoter.

9. The method of claim 8, wherein the promoter is small chicken beta-actin, EF1, or a tissue-specific promoter.

10. The method of claim 5, wherein the vector further encodes a detectable signal for quantifying expression of Msr.

11. The method of claim 10, wherein the detectable signal is a FLAG epitope sequence, T2A, a fluorescent moiety, Myc, or P2A.

12. The method of claim 5, wherein the vector comprises about 80% similarity or more to SEQ ID NO: 1 or SEQ ID NO: 2.

13. The method of claim 5, wherein the vector is encapsulated by a vehicle, and wherein the method further comprises delivering said vehicle to the subject.

14. The method of claim 13, wherein the vehicle is a nanoparticle.

15. The method of claim 5, wherein the vector is transfected into a cell, and wherein the method further comprises delivering said cell to the subject.

16. The method of claim 14, wherein the cell is derived from the subject.

17. The method of claim 5, wherein the retinal tissue damage is caused by oxidative stress.

18. The method of claim 5, wherein the vector is administered in a dose of from about 10 μL to about 150 μL.

19. The method of claim 5, wherein the vector is administered intraocularly or via eye drops.

20. The method of claim 5, wherein the vector is administered to retinal pigment epithelium (RPE) of the subject.
